(19) United States
(12) Patent Application Publication
(10) Pub. No.: US 2019/0385723 A1
(43) Pub. Date: Dec. 19, 2019

(54) MEDICATION SUPPORT APPARATUS, METHOD, AND PROGRAM

(71) Applicants: OMRON Corporation, Kyoto (JP); OMRON HEALTHCARE CO.,LTD., Kyoto (JP)

(72) Inventors: Hironori SATO, Kyoto (JP); Mitsuharu Konishi, Kyoto (JP); Seisuke Fujiwara, Kyoto (JP); Daisuke Nozaki, Kyoto (JP)

(73) Assignees: OMRON Corporation, Kyoto (JP); OMRON HEALTHCARE CO.,LTD., Kyoto (JP)

(21) Appl. No.: 16/554,918

(22) Filed: Aug. 29, 2019

Related U.S. Application Data
(63) Continuation of application No. PCT/JP2018/004911, filed on Feb. 13, 2018.

(30) Foreign Application Priority Data
Mar. 14, 2017 (JP) 2017-049176

(51) Int. Cl. G16H 20/10; A61B 5/00; A61B 5/021; G16H 40/63

(52) U.S. Cl. CPC G16H 20/10; A61B 5/0004; G16H 40/63; A61B 5/4833; A61B 5/742; A61B 5/021

(57) ABSTRACT

An apparatus includes a generation unit and a transmission unit. The generation unit generates, for a patient, first information indicating a status of drug compliance based on a acquired drug-taking information, and generates second information to display types and amounts of medicine included in medication information, transition of blood pressure value based on blood pressure measurement information, and the generated first information in a time line in association with one another, based on information indicating medication date and time, measurement date and time, and drug-taking date and time respectively included in the medication information, the blood pressure measurement information, and the drug-taking information. The transmission unit transmits the generated second information to a terminal.

### CROSS-REFERENCE TO RELATED APPLICATIONS

This application is a Continuation Application of PCT Application No. PCT/JP2018/004911, filed Feb. 13, 2018 and based upon and claiming the benefit of priority from Japanese Patent Application No. 2017-049176, filed Mar. 14, 2017, the entire contents of all of which are incorporated herein by reference.

### FIELD

Embodiments described herein relate generally to a medication support apparatus, a method, and a program for assisting a doctor's medication work, for example.

### BACKGROUND

In the treatment of high blood pressure, in general, a doctor prescribes an antihypertensive drug to a patient based on the examination result, and the patient takes a prescribed antihypertensive drug, for example. In addition, the patient uses the blood pressure monitor to measure the blood pressure at a fixed time each day and report this measurement result to the doctor at the next visit.

In addition, for example, a remote medical care system has been proposed which performs medical care using a network for patients in remote places. In this system, for example, a patient transfers vital measurement information such as blood pressure to a server via the Internet and registers the same on a home page Then, the doctor views the vital measurement data registered on the home page of the above server from his or her own terminal, writes a comment for the patient, and provides this comment to the patient's vital measurement apparatus at the next vital measurement. (Refer to, for example, Jpn. Pat. Appln. KOKAI Publication No. 2002-312487).

However, even in outpatient clinics or telemedicine clinics, doctors generally grasp the treatment results of the patient with the use of the blood pressure value declared or measured by the patient as a judgment material. Therefore, for example, each time the blood pressure value of the patient rises or falls and there arises the necessary to change the prescription of the antihypertensive drug accordingly, the doctor needs to read the patient's charts to check the past medication prescriptions. Also, in order to change the medication prescription, it is necessary to check whether the patient has taken the antihypertensive drug as prescribed. In general, however, the patient verbally explains his or her rough drug-taking status to the doctor at the time of medical treatment. Thus, the doctor may not be able to accurately determine the relationship between the transition of blood pressure value and the drug-taking status.

### SUMMARY

A first aspect of the present invention is a medication support apparatus that is capable of communicating via a network between a first terminal used by a doctor and a second terminal including a blood pressure monitor used by a patient. The medication support apparatus acquires medication information including types and amounts of medicine prescribed for the patient from the first terminal via the network and acquires blood pressure measurement information of the patient obtained by the blood pressure monitor from the second terminal via the network. In addition, for each patient, a display information generation unit generates medication treatment result information to display the types and amounts of medicine included in the acquired medication information and transition of blood pressure value based on the acquired blood pressure measurement information in a time line in association with each other, based on information indicating medication date and time and measurement date and time respectively included in the medication information and the blood pressure measurement information. Then, upon receipt of a display request for the medication treatment result information for the patient from the first terminal, the medication treatment result information corresponding to the patient is transmitted to the first terminal as the request source via the network.

According to the first aspect, in the medication support apparatus, for each patient, the medication treatment result information is generated to display the types and amounts of medicine included in the medication information acquired from a terminal of a prescriber such as a doctor and the transition of the blood pressure value based on the blood pressure measurement information acquired from the terminal of the patient in the time line in association with each other, and the medication treatment result information is transmitted and presented to the terminal of the prescriber as the request source. This makes it possible to present the medication treatment result information without being generated by the terminal of the prescriber, thereby to reduce a processing load on the terminal of the prescriber. In addition, there is no need to install a special application for generating the medication treatment result information on the terminal of the prescriber, whereby the prescriber can carry out the invention without new expense burden.

Furthermore, when performing outpatient treatment or remote treatment for a patient, the prescriber such as a doctor can grasp the transition of the blood pressure value of the patient in the past in association with the types and amounts of medicine prescribed in the past, simply by viewing the displayed medication treatment result information. Therefore, the prescriber can make an efficient and accurate determination on medication for the patient, that is, on the need to change medicine and the details of the change.

In a second aspect of the present invention, the display information generation unit detects a change exceeding a preset amount of the blood pressure value from the transition of the blood pressure value based on the blood pressure measurement information, and generates the medication treatment result information to display information indicating details of the change to be added to at a corresponding position in a time line of a graph indicating the transition of the blood pressure.

According to the second aspect, a change exceeding the preset amount of the blood pressure value is detected from the transition of the blood pressure value based on the blood pressure measurement information, and the information indicating the details of the change is displayed on the graph of the transition of the blood pressure at the corresponding time position. Accordingly, the doctor can recognize abnormal changes in blood pressure values at a glance, and makes a more efficient and accurate determination on medication.

In a third aspect of the present invention, the display information generation unit generates the medication treatment result information to display the types of the medicine included in the medication information as blocks different in color or gray scale and display the amounts of the medicine by the number of stacks of blocks.

According to a third aspect, the type of medicine is displayed as different blocks in color or grayscale, and the amount of medicine is displayed by the number of stacked blocks. Accordingly, the types and amounts of medicines administered in the past can be grasped easily and accurately by the colors and numbers of the blocks. This makes it possible to make a further efficient and accurate determination on medication.

A fourth aspect of the present invention further includes a drug-taking information acquisition unit that acquires drug-taking information indicating drug-taking status of the patient from the second terminal via the network. In addition, the display information generation unit generates drug-taking adherence information indicating a state of drug compliance based on the acquired drug-taking information, and generates the medication treatment result information to display the generated drug-taking adherence information in a time line in association with information indicating the types and amounts of the medicine and the transition of the blood pressure value.

According to the fourth aspect of the present invention, the drug-taking adherence information indicating the status of drug compliance is generated based on the drug-taking information acquired from the terminal of the patient, and the drug-taking adherence information is displayed in the time line in association with the types and amounts of the medicine and the transition of the blood pressure value. Accordingly, the transition of the blood pressure and the drug-taking adherence can be considered as requirements of determination on medication for the patient, which allows a further efficient and accurate determination on medication.

A fifth aspect of the present invention further includes an activity amount information acquisition unit that acquires activity amount information indicating an activity amount of the patient from the second terminal via the network. In addition, the display information generation unit generates information indicating transition of the activity amount based on the acquired activity amount information, and generates the medication treatment result information to display the generated information indicating the transition of the activity amount in a time line in association with the types and amounts of the medicine and the transition of the blood pressure value.

According to the fifth aspect of the present invention, the information indicating the transition of the activity amount is generated based on the activity amount information acquired from the terminal of the patient, and the information indicating the transition of the activity amount is displayed in the time line in association with the types and amounts of the medicine and the transition of the blood pressure value. Accordingly, the activity amount can be considered as a requirement of determination on medication for the patient, which allows a further efficient and accurate determination on medication.

According to a sixth aspect of the present invention, when receiving a medication support request for the patient from the first terminal, at least the medication treatment result information corresponding to the patient is referred to generate medication guide information indicating candidates for types and amounts of medicine to be prescribed to the patient, and the generated medication guide information is transmitted to the first terminal as the request source via the network.

According to the sixth aspect, in response to the doctor's request, the medication guide information indicating the candidates for the types and amounts of the medicine is generated in consideration to at least the medication treatment result information corresponding to the patient to be treated, and is displayed on the terminal of the doctor. Accordingly, when vacillating on the types and amounts of medicine to be prescribed, the doctor can refer to the medication guide information to make a determination on the details of the medication.

A seventh aspect of the present invention is a medication support apparatus that is capable of communicating via a network between a first terminal used by a prescriber of medicine and a second terminal that is connected to a blood pressure monitor or contains the blood pressure monitor and is used by a patient. The medication support apparatus acquires medication information including types and amounts of medicine prescribed for the patient from the first terminal via the network, acquires blood pressure measurement information of the patient obtained by the blood pressure monitor from the second terminal via the network, and acquires drug-taking information indicating a drug-taking status of the patient from the second terminal via the network. In addition, for each of the patients, the medication support apparatus generates drug-taking adherence information indicating a status of drug compliance based on the acquired drug-taking information, generates medication treatment result information to display the generated drug-taking adherence information and the types and amounts of medicine included in the acquired medication information in a time line in association with each other based on medication date and time and drug-taking date and time respectively included in the medication information and the drug-taking information, and transmits the medication treatment result information to the first terminal as the request source via the network.

According to the seventh aspect of the present invention, the drug-taking adherence information indicating the status of drug compliance is generated based on the drug-taking information acquired from the terminal of the patient, and the drug-taking adherence information is displayed in the time line in association with information indicating the transition of the types and amounts of the medicine. Accordingly, the drug-taking adherence can be considered as requirements of determination on medication for the patient, which allows a further efficient and accurate determination on medication in consideration to the drug-taking status of the patient.

According to the aspects of the present invention, it is possible to provide a technique by which to present information indicating at least blood pressure transition or the relationship between drug-taking status and medication without being generated by a terminal of a prescriber, thereby to reduce a processing load on the terminal of the prescriber.

In addition, it is possible to provide a technique to allow a prescriber to check at least the transition of the blood pressure or the relationship between the drug-taking status and medication, thereby offering accurate medication.

### BRIEF DESCRIPTION OF THE DRAWING(S)

FIG. 1 is a diagram illustrating an overall configuration of a system including a medication support apparatus according to a first embodiment of the present invention.

FIG. 2 is a block diagram illustrating a functional configuration of the medication support apparatus according to the first embodiment of the present invention.

FIG. 3 is a flowchart illustrating a processing procedure and processing contents of medication support control by the medication support apparatus illustrated in FIG. 2.

FIG. 4 is a diagram illustrating an example of medication treatment result display data generated by the medication support control illustrated in FIG. 3.

FIG. 5 is a diagram illustrating another example of medication treatment result display data generated by the medication support control illustrated in FIG. 3.

### DETAILED DESCRIPTION

Hereinafter, embodiments according to the present invention will be described with reference to the drawings.

An object of the present embodiments is to provide a technique by which to present information indicating at least blood pressure transition or the relationship between drug-taking status and medication without being generated by a terminal of a prescriber, thereby to reduce a processing load on the terminal of the prescriber.

Another object of the present embodiments is to provide a technique for allowing the prescriber to grasp transition in the patient's blood pressure in the past in association with types and amounts of medicines prescribed in the past so that the subscriber can make an efficient and accurate determination on medication for the patient.

### First Embodiment

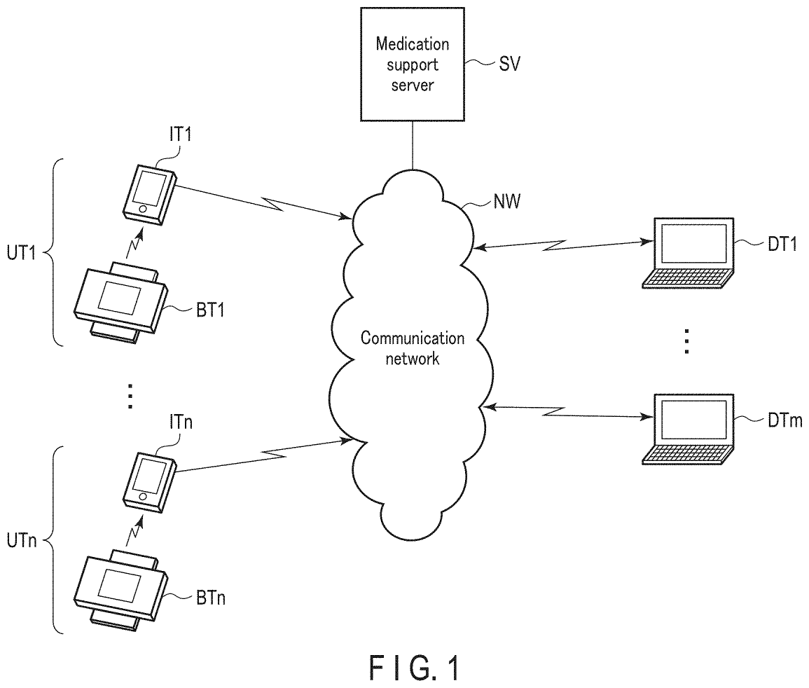

FIG. 1 is a diagram illustrating an overall configuration of a medication support system according to a first embodiment. In FIG. 1, SV represents a medication support server that functions as a medication support apparatus, DT1 to DTm represent first terminals used by doctors (hereinafter also referred to as doctor terminals), and UT1 to UTn represent second terminals used by patients (also referred to as patient terminals). The medication support server SV, the doctor terminals DT1 to DTm, and the patient terminals UT1 to UTn can communicate with each other via a communication network NW.

Each of the doctor terminals DT1 to DTm includes, for example, a stationary personal computer, a portable notebook personal computer, and a tablet or wearable portable information terminal. Each of the doctor terminals DT1 to DTm includes at least a browser that can be used to access the medication support server SV.

The patient terminals UT1 to UTn include respectively blood pressure monitors BT1 to BTn and portable information terminals IT1 to ITn. The blood pressure monitors BT1 to BTn are attached to the wrist of a patient, for example, to measure blood pressure by oscillometric methods, for example, by the operation of the patient or at preset timings or time intervals, and wirelessly transmit the blood pressure data via a wireless interface to the portable information terminals IT1 to ITn. The measurement method with the blood pressure monitors BT1 to BTn may be a beat-by-beat method by which to measure the blood pressure on a beat-by-beat basis or a trigger measurement method by which to estimate blood pressure fluctuation by a pulse transit time (PTT) method and sporadically measure the blood pressure with the estimated fluctuation as a trigger. Further, the blood pressure monitors BT1 to BTn are not limited to the wrist-attached type, but may be a type with a cuff to be wound around the upper arm or a stationary type.

The portable information terminals IT1 to ITn are smartphones, tablet-type or wearable terminals, for example. Each of the portable information terminals IT1 to ITn has a blood pressure data transfer unit and a patient data transmission unit. Each of the blood pressure data transfer units receives the blood pressure data of the patient transmitted from the blood pressure monitors BT1 to BTn, and transfer the blood pressure data to the medication support server SV via the communication network NW. Each of the patient data transmission units transmits information indicating the drug-taking status manually input by the patient, information indicating the activity amount, information indicating the body weight of the patient, and information indicating the meal intake status to the medication support server SV via the communication network NW. As a wireless interface used between the blood pressure monitors BT1 to BTn and the portable information terminals IT1 to ITn, an interface under a near-field wireless data communication standard such as Bluetooth (registered trademark) is used.

When the blood pressure monitors BT1 to BTn do not have a function to transmit blood pressure data, the portable information terminals IT1 to ITn transmit information indicating blood pressure values and measurement dates and times manually input by the patients to the medication support server SV.

When each of the portable information terminals IT1 to ITn is a wearable terminal to be attached to the patient's wrist, the wearable terminal contains a blood pressure measuring unit, an activity amount measurement unit, a step counting unit, a sleep state measurement unit, and an environmental measurement unit to measure temperature, humidity, air pressure, and others. Then, the wearable terminal automatically transmits the measurement data obtained by the measurement units, at a preset timing or periodically. The measurement values of the activity amount, the number of steps, and the sleep state can be calculated based on the measurement data of a triaxial acceleration sensor. In addition, the temperature, humidity, and pressure can be determined by the measurement data of a thermometer, a hygrometer, and a barometer, respectively.

When an application program for managing the patient's health conditions and living conditions is installed in the portable information terminals IT1 to ITn, the portable information terminals IT1 to ITn can provide the application program with the measurement data of the blood pressure, the activity amount, the number of steps, the sleep state, the environment, and others. The measurement data can be provided by including a data connect function in the portable information terminals IT1 to ITn. The data connect function is intended to perform authentication between the application program and each of the measurement data or the measurement units, for example. When the application program is qualified to use the measurement data, the data connect function transfers the measurement data to the application program. The data connect function may be allowed to convert the measurement data into a data format that can be handled by the application program.

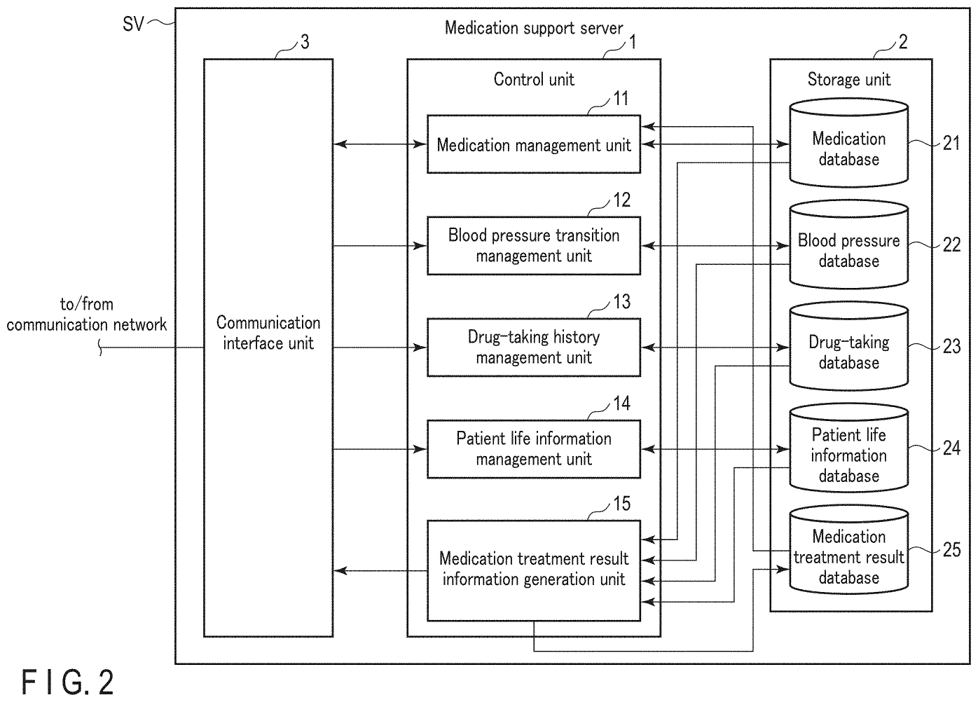

The medication support server SV includes a server computer and is configured as described below. FIG. 2 is a block diagram illustrating a functional configuration of the medication support server SV.

That is, the medication support server SV includes a control unit 1, a storage unit 2, and a communication interface unit 3.

The communication interface unit 3 performs data communication between the doctor terminals DT1 to DTm and the portable information terminals IT1 to ITn via the communication network NW under the control of the control unit 1. As a communication protocol, a protocol defined in the communication network NW is used. Specifically, Transmission Control Protocol/Internet protocol (TCP/IP), User Datagram Protocol/Internet protocol (UDP/IP), or the like is used.

Used for the storage unit 2 as storage media are a non-volatile memory such as a hard disk drive (HDD) or a solid state device (SSD) that can be written and read any time and a volatile memory such as a random access memory (RAM) that can be written and read any time. The storage unit 2 has a medication database 21, a blood pressure database 22, a drug-taking database 23, a patient life information database 24, and a medication treatment result information database 25, for example, as databases necessary for carrying out the first embodiment. The storage unit 2 is also provided with a storage area as a program memory.

The medication database 21 is used to store medicine information, medication guide information, and medication management information. The medicine information includes types (classes) of medicines and information on indications and contraindications by type. The medication guide information indicates the types and amounts of medicine to be prescribed to the patient, and has a plurality of candidates for the types and amounts according to a combination of the type and amount of antihypertensive drug in the prescription, transition of blood pressure value, changes in activity amount, and the patient's weight. The medication management information is created for each patient and includes associations between the types and amounts of medicine prescribed for the patient and the prescribed dates.

The blood pressure database 22 is used to store information indicating the transition of the blood pressure data for each patient. The drug-taking database 23 is used to store information indicating the medication history of each patient. The patient life information database 24 is used to store information indicating the activity amount, body weight, and meal intake status of each patient, that is, patient life information. The medication treatment result information database 25 is used to store medication treatment result information generated by the control unit 1 described later for each patient.

The control unit 1 includes a hardware processor called a central processing unit (CPU) and a working memory, for example. As processing function units necessary for carrying out the first embodiment, the control unit 1 has a medication management unit 11, a blood pressure transition management unit 12, a drug-taking history management unit 13, a patient life information management unit 14, and a medication treatment result information generation unit 15. All these processing function units are implemented by causing the hardware processor to execute programs stored in the program memory of the storage unit 2. That is, the hardware processor is configured to operate each of the processing function units.

The medication management unit 11 has a medication guide transmission unit and a medication information management unit. Upon receipt of a request for transmission of medication guide from the doctor terminals DT1 to DTm, the medication guide transmission unit reads the medication treatment result information of the applicable patient from the medication treatment result information database 25. Then, based on the read medication treatment result information, the medication guide transmission unit selects candidates for the types and amounts of medicine to be prescribed from the medication database 21, and transmits medication guide information including the selected candidates for the types and amounts of medicines to be prescribed to the doctor terminals DT1 to DTm as the request source.

Each time electronic medical record (EMR) data of the patient is sent from the doctor terminals DT1 to DTm, the medication information management unit extracts the medication information (prescription information) from the electronic medical record data and adds the prescription information together with information indicating the medication date and time to the medication management information of the patient stored in the medication database 21.

Each time blood pressure data is sent from the patient terminals UT1 to UTn, the blood pressure transition management unit 12 causes the blood pressure database 22 to store the blood pressure data in association with patient identification information together with time stamp information indicating the measurement date and time. That is, the blood pressure transition management unit 12 updates and stores the information indicating the transition of the blood pressure of each patient.

Each time information indicating the drug-taking status is sent from the patient terminals UT1 to UTn, the drug-taking history management unit 13 stores the information indicating the drug-taking status together with information indicating the measurement date and time in association with identification information of the patient in the drug-taking database 23, and calculates the drug-taking adherence based on the information indicating the stored drug-taking status.

Each time information indicating activity amount, body weight, and meal intake status is sent from the patient terminals UT1 to UTn, the patient life information management unit 14 stores the information indicating the activity amount, the body weight, and the meal intake status together with information indicating measurement date and time or input date and time in association with the identification information of the patient in the drug-taking database 23 as patient life information.

The medication treatment result information generation unit 15 reads, for each patient, the medication management information, the blood pressure transition information, the drug-taking adherence information, and the patient life information, respectively, from the medication database 21, the blood pressure database 22, the drug-taking database 23, and the patient life information database 24 in a pre-specified cycle. Then, the medication treatment result information generation unit 15 generates information indicating the medication treatment result based on the read information, and stores the generated medication treatment result information in association with the identification information of the patient in the medication treatment result information database 25.

Further, upon receipt of a request for transmission of the medication treatment results from the doctor terminals DT1 to DTm, the medication treatment result information generation unit 15 reads the medication treatment result information of the patient specified by the request from the medication treatment result information database 25, and transmits the medication treatment result information to the doctor terminals DT1 to DTm as the request source.

### (Operations)

Next, an operation example of the medication support server SV configured as described above will be described.

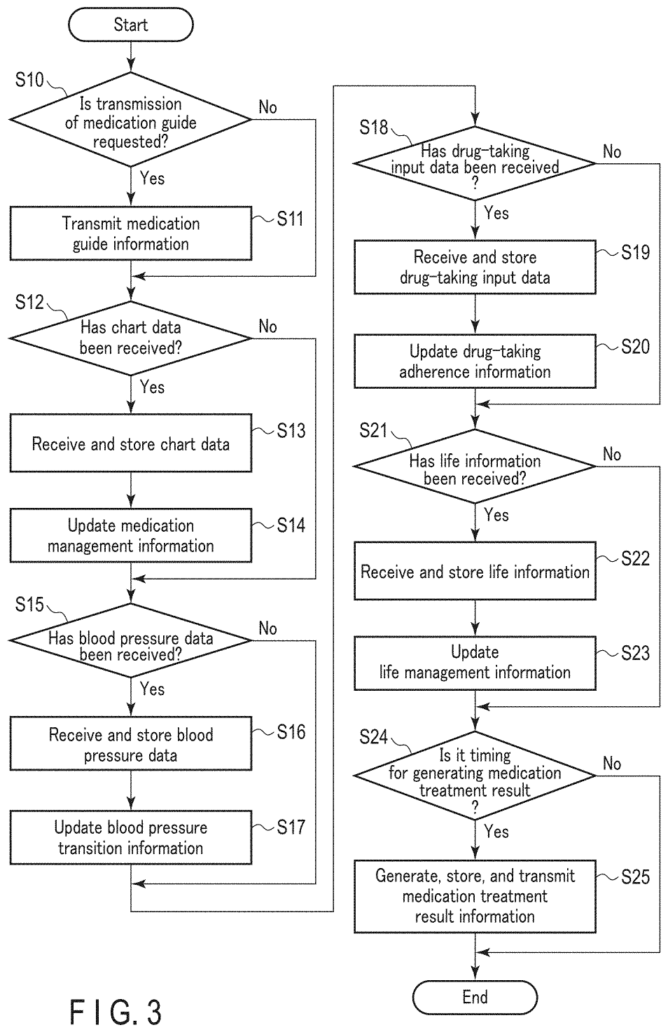

FIG. 3 is a flowchart illustrating the processing procedure and processing contents of the medication support server SV.

The description here will be provided taking the case of a re-examination patient rather than a new patient as an example, on the assumption that medicine (antihypertensive drugs) was prescribed at the time of the previous treatment or earlier and, after that, blood pressure data, information indicating the drug-taking status, and patient life information such as activity amount, body weight, and meal-intake status have been received from the portable information terminals IT1 to ITn of the patient.

### (1) Provision of a Medication Guide

In the case of a re-examination patient, the medication support server SV generates medication treatment result information based on the type and amount of an antihypertensive drug in prescription, received blood pressure data, information indicating drug-taking status, and patient life information, and the medication treatment result information is stored in the medication treatment result information database 25. In addition, the specific example of medication treatment result information will be described in detail later.

If it is necessary to change the prescription of antihypertensive drugs for the re-examined patient and determine the contents of the change, the doctor specifies the identification information of the patient at his or her doctor terminals DT1 to DTm, and then inputs a command to request for transmission of the medication guide. Then, the medication guide transmission request including the identification information of the patient is transmitted from the doctor terminals DT1 to DTm to the medication support server SV.

The medication support server SV monitors for the receipt of the medication guide transmission request in step S10 in the standby state. In this state, upon receipt of the medication guide transmission request, the medication support server SV generates medication guide information as described below under the control of the medication management unit 11 in step S11.

That is, the medication management unit 11 reads corresponding medication treatment result information from the medication treatment result information database 25 based on the identification information of the specified patient. Then, the medication management unit 11 searches the medication database 21 based on the read medication treatment result information. At this time, stored in the medication database 21 is a plurality of combinations of preset candidates for prescription of the types and amounts of antihypertensive drugs corresponding to combinations of the types and amounts of antihypertensive drugs in prescription, the transition of blood pressure value, change in the activity amount, and the body weight of the patient. Accordingly, the candidates for prescription of the types and amounts of antihypertensive drugs corresponding to the medication treatment result information of the patient are read from the medication database 21.

For example, it is assumed that, in the first step of treatment, the blood pressure value is increased by 15 mmHg or more in the state in which one tablet of angiotensin converting-enzyme inhibitor (ACE) inhibitor and one tablet of Ca antagonist (calcium channel blocker (CCB)) are prescribed, and the drug-taking adherence (Adherence) and activity amount (Activity) are equal to greater than predetermined values. In this case, a candidate for prescription “add one unit of diuretic (DIU)” is read out from the medication database 21. When the weight of the patient is equal to or more than a predetermined value, a candidate for prescription “add two units of diuretic (DIU)” is read out.

The candidates for the prescription described above can be determined in a more accurate manner by referring to the hypertension treatment guidelines and information indicating the patient's previous disease. The hypertension treatment guidelines define a prescription plan that indicates the type and amount of medicine to be prescribed to a patient at each stage of treatment. For example, in the case of hyperpiesia, the treatment guidelines are determined in consideration to blood pressure value and its transition (drug-taking effect), presence or absence of complications, the body weight of the patient, and others. The hypertension treatment guidelines are described, for example, in Japan Hypertension Treatment Guidelines 2014 (JSH 2014).

The medication management unit 11 transmits the read candidates for prescription of the type and amount of the antihypertensive drug as medication guide information to the doctor terminals DT1 to DTm as the request source. As a result, the medication guide information is displayed on the doctor terminals DT1 to DTm. The doctor decides whether there is the need to change the prescription of the antihypertensive drug and the contents of the change with reference to the displayed medication guide information and in consideration to the presence or absence of side effects due to the ACE inhibitor.

The doctor terminals DT1 to DTm transmit the prescription decided by the doctor to the medication support server SV, and the medication management unit 11 of the medication support server SV may update the contents of the applied medication guide information stored in the medication database 21 based on the contents of the prescription decided by the doctor. In addition, the medication management unit 11 may not update the contents of the medication guide information but may add the contents of the prescription decided by the doctor as a new candidate.

### (2) Management of Medication Information

When completing the medical treatment of the patient and updating the contents of the electronic medical chart, the doctor transmits the data of the electronic medical record from his or her doctor terminals DT1 to DTm to the medication support server SV. The medication support server SV monitors for the receipt of the electronic medical chart data in step S12 in the standby state. Then, upon receipt of the electronic medical chart data, the medication support server SV stores the received electronic medical chart data in the medication database 21 in association with the identification information of the patient under the control of the medication management unit 11 in step S13.

Next, in step S14, the medication management unit 11 extracts medication information, that is, prescription information indicating the type and amount of medicine prescribed to the patient, from the electronic medical chart data, and adds the extracted prescription information to the medication management information of the applicable patient in the medication database 21.

Thereafter, each time electronic medical chart data is received from the doctor terminals DT1 to DTm, the medication management unit 11 repeatedly executes steps S12 to S14.

### (3) Management of Blood Pressure Data

At the patient terminals UT1 to UTn, the blood pressure of the patient in the home or office is measured by the blood pressure monitors BT1 to BTn, for example, at fixed time intervals. Then, the measured blood pressure data is sent from the blood pressure monitors BT1 to BTn to the portable information terminals IT1 to ITn, and is transmitted from the portable information terminals IT1 to ITn to the medication support server SV.

The medication support server SV monitors for the receipt of blood pressure data in step S15 in the standby state. Then, when the blood pressure data is transmitted from the portable information terminals IT1 to ITn of the patient, the blood pressure transition management unit 12 receives the blood pressure data in step S16, and then additionally stores the blood pressure data together with time stamp information indicating measurement date and time in association with the identification information of the patient in the blood pressure database 22.

Next, in step S17, the blood pressure transition management unit 12 calculates the weekly average of blood pressure values measured during the period every week, for example, based on the blood pressure data stored in the blood pressure database 22, and stores the calculated value in the blood pressure database 22 as blood pressure transition information of the patient. Further, the blood pressure transition management unit 12 compares the blood pressure average value of this week with the blood pressure average value of the previous week based on the foregoing blood pressure transition information to determine whether the change amount is equal to or more than a threshold. The threshold for change amount is set to a value medically defined as an abnormal blood pressure change. Then, when an abnormal blood pressure change is detected, the blood pressure transition management unit 12 adds information indicating the amount of the change and the change direction (increase or decrease) to the blood pressure average value of this week.

Thereafter, at each receipt of new blood pressure data, the blood pressure transition management unit 12 repeatedly executes steps S15 to S17.

### (4) Management of Drug-Taking Status

The patient takes the prescribed antihypertensive drug in the specified regimen. Then, each time he or she takes the drug, the patient inputs a report to that effect along with the drug-taking date and time to his or her own portable information terminals IT1 to ITn. If he or she forgets to take the drug, the patient inputs a report to that effect along with the date and time when the drug should have been taken. The portable information terminals IT1 to ITn transmit the input information indicating the presence or absence of drug taking and the date and time to the medication support server SV as medication input data.

The medication support server SV monitors for the receipt of input drug-taking data in step S18 in the standby state. Then, when the input drug-taking data is transmitted from the portable information terminals IT1 to ITn of the patient, the drug-taking history management unit 13 receives the input drug-taking data in step S19, and then additionally stores the input drug-taking data in association with the identification information of the patient in the drug-taking database 23.

Next, in step S20, the drug-taking history management unit 13 calculates, based on the input drug-taking data stored in the drug-taking database 23 drug-taking adherence representing the execution degree of drug-taking in the applicable period, for example, every week, and stores the calculated drug-taking adherence information in the drug-taking database 23.

Thereafter, at each receipt of new input drug-taking data, the drug-taking history management unit 13 repeatedly executes steps S18 to S20.

### (5) Management of Patient Life Information

The patient inputs every day his/her activity amount data in home or office and information indicating his/her body weight and meal intake status together with the measurement date and time to the portable information terminals IT1 to ITn. The activity amount data is represented by the number of steps measured by the pedometer or the amount of motion measured by the acceleration sensor, for example. The portable information terminals IT1 to ITn transmit the input information as patient life information to the medication support server SV. The activity amount data may be manually input by the patient, or may be measured by an activity amount measurement application so that the measurement data may be input.

The medication support server SV monitors for the reception of patient life information in step S21 in the standby state. Then, when patient life information is sent from the portable information terminals IT1 to ITn of the patient, the patient life information management unit 14 receives the patient life information at step S22, and then additionally stores the patient life information in association with the identification information of the patient in the patient life information database 24.

Subsequently, in step S23, for each patient, the patient life information management unit 14 calculates the weekly average of the activity amount and the body weight on a weekly basis, and stores the results in the patient life information database 24. Thereafter, each time new patient life information is received, the patient life information management unit 14 repeatedly executes steps S21 to S23.

### (6) Generation of Medication Treatment Result Information

In the standby state, the medication support server SV monitors whether it is time to generate medication treatment result information in step S24. The generation timing is set, for example, every week. The medication support server SV activates the medication treatment result information generation unit 15 in step S25 each time the above generation timing is reached. Then, under the control of the medication treatment result information generation unit 15, the medication treatment result information is generated as described below, and the result is stored in the medication treatment result information database 25 in association with the identification information of the patient.

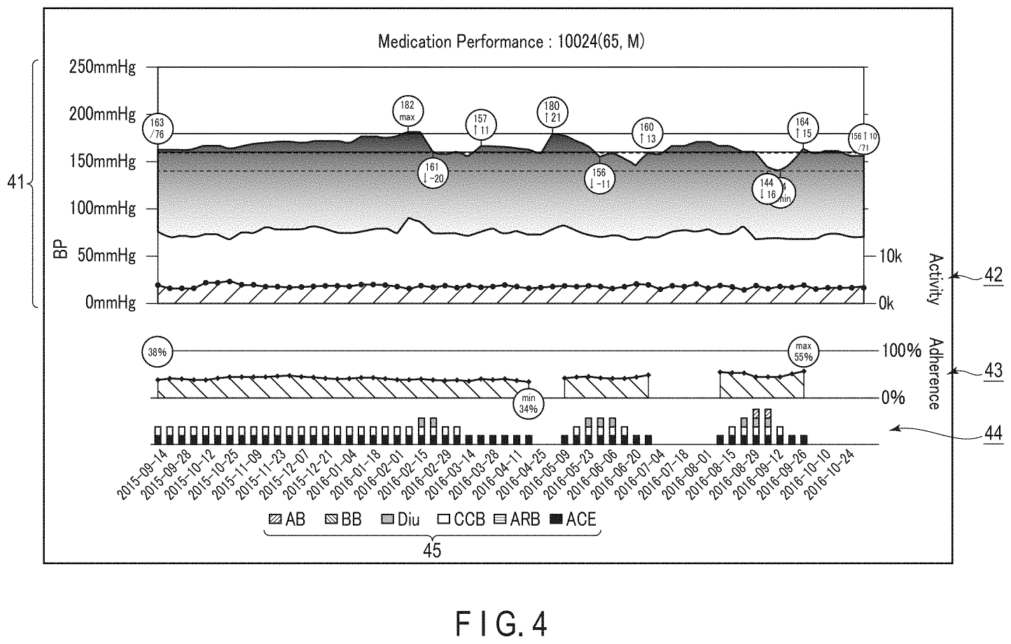

Specifically, the medication treatment result information generation unit 15 first reads, for each patient, the medication management information, the blood pressure transition information, the drug-taking adherence information, and the patient life information, respectively, from the medication database 21, the blood pressure database 22, the drug-taking database 23, and the patient life information database 24. Next, the medication treatment result information generation unit 15 produces medication management information in which the pieces of read information are arranged in the vertical direction with the time axis being made coincident. FIG. 4 is a diagram illustrating an example of the medication management information.

As illustrated in FIG. 4, blood pressure (BP) transition 41, activity amount change (Activity) 42, and drug-taking adherence (Adherence) 43 are all represented by line graphs. In the background of the line graph representing the blood pressure transition, a band-like region indicating a high blood pressure stage is displayed, so that it can be checked at a glance whether the change in blood pressure of the patient is in the high blood pressure stage. In addition, in the line graph representing the blood pressure transition, the abnormal value (outlier value) of blood pressure and the information indicating the amount of change and the direction of change are superimposed at the time position at which the abnormal value was measured. As a result, the doctor can confirm the abnormal value of blood pressure and the amount of change and the direction of change at a glance in association with the measurement date and time.

The activity amount change 42 is displayed as a value obtained by converting the activity amount into a weekly average value of movement distance km, for example. The drug-taking adherence 43 is represented by the weekly average value [%] of the degree of taking the drug. In addition to or instead of the activity amount (Activity), other indicators related to the activity amount, for example, the body weight and the movement distance or the number of steps may be displayed.

On the other hand, when the medication management information 44 is displayed, the types of antihypertensive drugs are represented as blocks different in color or grayscale, and the amounts of antihypertensive drugs are represented by the number of stacked blocks. In the example illustrated in FIG. 4, one tablet of ACE inhibitor and one tablet of Ca antagonist (CCB) were prescribed in the period until Feb. 15, 2016, but, due to an increase in the blood pressure value, one tablet of diuretic (DIU) was prescribed in addition to the ACE inhibitor and the Ca antagonist (CCB) from Feb. 15 to Feb. 29, 2016.

As a result, a drop in the blood pressure value was recognized, and thus the prescription was returned to only the ACE inhibitor and the Ca antagonist (CCB) from Feb. 29 to Mar. 14, 2016, and then the prescription was changed to only the ACE inhibitor from Mar. 14 to Apr. 25, 2016. Further, the medication was stopped in the period from Apr. 25, to May 9, 2016 and in the period from Jul. 4 to Aug. 8, 2016.

In addition, in the period from Aug. 29 to Sep. 12, 2016, in addition to ACE inhibitor, an alpha blocker (AB) was prescribed in addition to the Ca antagonist (CCB) and the diuretic (DIU). Although not illustrated in FIG. 4, when the amount of the Ca antagonist (CCB) is increased to two tablets, for example, the increase is indicated by two stacked blocks representing the Ca antagonist (CCB). The block may have any shape such as a rectangle, a circle, or a cube as far as it is closed.

In addition, the medication treatment result information generation unit 15 calculates the achievement score for the target value on a weekly basis, for each of the medication management information, the blood pressure transition information, the drug-taking adherence information, and the patient life information, respectively read from the medication database 21, the blood pressure database 22, the drug-taking database 23, and the patient life information database 24.

For example, for the blood pressure (BP) and the body weight (Body Fat), a target value X_target, a latest measurement value is X_now, and a past measurement value X_prev are calculated as

1−(X_target−X_now)/(X_target−X_prev).

For the drug-taking adherence (Adherence) and the activity amount (Activity), the target value and the measurement values are calculated as

(X_now−X_prev)/(X_target−X_prev).

For the meal intake status (meal), an actual number of meals in a week as X, a maximum number of withdrawals as negative max, and a maximum number of meals as positive max are calculated as

(X+negative max)/(positive max+negative max).

Next, the medication treatment result information generation unit 15 calculates an overall achievement score X based on an achievement score X1 of the blood pressure (BP), an achievement score X2 of the body weight (Body Fat), an achievement score X3 of the drug-taking adherence (Adherence), an achievement score X4 of the activity amount (Activity), and an achievement score X5 of the meal intake status (meal) as

X=(W1X1+W2X2+ . . . +W5X5)/(W1+W1+ . . . +W5)

where W1, W2, . . . , W5 are coefficients.

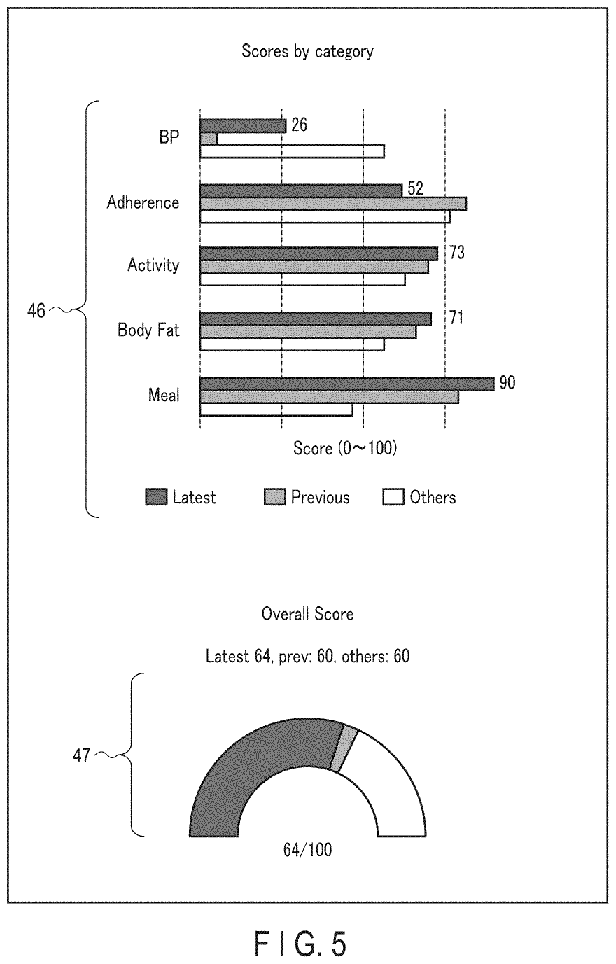

Then, the medication treatment result information generation unit 15 displays the calculated achievement scores by category of this week, that is, the achievement score X1 of the blood pressure (BP), the achievement score X2 of the body weight (Body Fat), the achievement score X3 of the drug-taking adherence (Adherence), the achievement score X4 of the activity amount (Activity), and the achievement score X5 of the meal intake status (meal), aligned with the achievement scores of the previous week and the average values of the achievement scores of a group of other patients of this week. Along with that, the calculated overall achievement score X is displayed by a semi-circle graph. FIG. 5 illustrates an example of the score display, in which the number 46 indicates the achievement scores by category, and the number 47 indicates the overall achievement score X.

The information illustrated in FIGS. 4 and 5 may be data displayed on different pages, but may be data displayed on one page so that the data can be viewed in a list.

### (7) Output of the Medication Treatment Result Information

Upon receipt of a request for transmission of the medication treatment result information from the doctor terminals DT1 to DTm, the medication treatment result information generation unit 15 of the medication support server SV reads the medication treatment result information corresponding to the patient specified by the request for transmission from the medication treatment result information database 25. Then, the medication treatment result information generation unit 15 transmits the read medication treatment result information from the communication interface unit 3 to the doctor terminals DT1 to DTm as the request source.

As a result, in the doctor terminals DT1 to DTm, the medication treatment result information of the arbitrarily specified patient is displayed on the display, so that the doctor can check the medication treatment result of the patient from the medication treatment result information of the patient displayed on the list window.

In addition, the arrangement positions of the displayed indicators such as the blood pressure transition (BP) transition 41, the activity amount change (Activity) 42, the drug-taking adherence (Adherence) 43, and the medication management information 44 can be exchanged by the doctor dragging the mouse at the doctor terminal DT1 to DTn. That is, the doctor can customize the display screen of the medication treatment result information in an easy-to-see manner at his/her wish. The doctor can customize the display screen also in the case of displaying other indicators related to the activity amount, for example, the body weight, the movement distance, or the number of steps in addition to or instead of the activity amount (Activity).

In addition, when the doctor selects by a clicking operation any of icons 45 indicating the types (classes) of antihypertensive drugs displayed at the bottom of the medication management information 44, display of the applied antihypertensive drug in the medication management information 44 is switched on or off at the doctor terminal DT1 to DTn. In response to the selection operation of the antihypertensive drug, only the drug-taking adherence corresponding to the selected antihypertensive drug is displayed in the display area of the drug-taking adherence (Adherence) 43 at the doctor terminals DT1 to DTn. Therefore, the doctor can check the drug-taking adherence (Adherence) for each antihypertensive drug.

### Advantageous Effects of the First Embodiment

As described above in detail, in the first embodiment, the medication support server SV generates the medication treatment result information to align and display the blood pressure (BP) transition 41, the activity amount (Activity) change 42, the drug-taking adherence (Adherence) 43, and the medication management information 44 in the same time line at time positions associated with one another, and transmits the medication treatment result information to the doctor terminals DT1 to DTm for display. Therefore, the doctor terminals DT1 to DTm can simply receive and display the medication treatment result information without generating the medication treatment result information in their own terminals. That is, the doctor terminals DT1 to DTm can present the medication treatment result information to the patient under a light processing load without installing a new application.

In addition, when performing outpatient treatment or remote treatment for a patient, simply by viewing the medication treatment result information displayed on the doctor terminals DT1 to DTm, the doctor can grasp at a glance the transition of the blood pressure value of the patient in the past in association with the type and amount of the antihypertensive drug prescribed in the past, thereby to make an accurate determination on the medication. Furthermore, by viewing the medication treatment result information, the doctor can consider the drug-taking adherence and the activity amount as requirements for determination on medication for the patient. Accordingly, the doctor can make a further accurate determination on the need to change the type of the antihypertensive drug and the contents of the change.

In addition, when an abnormal value (outlier value) of blood pressure is detected, the medication support server SV generates the medication treatment result information in which the information indicating the abnormal value, the amount of a change from the previous measurement value, and the direction of the change is superimposed on a line graph indicating blood pressure transition at an applied time position, and displays the medication treatment result information on the doctor terminals DT1 to DTm. Accordingly, by viewing the medication treatment result information displayed on the doctor terminals DT1 to DTm, the doctor can recognize abnormal fluctuation in the blood pressure value at a glance, thereby making a further efficient determination on the medication.

Furthermore, the medication support server SV generates the medication treatment result information in which the types of antihypertensive drugs are represented by blocks different in color or gray scale and the amounts of the antihypertensive drugs are represented by the number of stacked blocks, and displays the medication treatment result information on the doctor terminals DT1 to DTm. Accordingly, by viewing the medication treatment result information displayed on the doctor terminals DT1 to DTm, the doctor can grasp the types and amounts of the prescribed antihypertensive drug by the color and number of the blocks, thereby making a further efficient determination on the next medication.

Moreover, when a plurality of types of antihypertensive drugs is prescribed simultaneously, the medication support server SV generates the medication treatment result information in which the display positions of the blocks are sorted and represented according to the selection order of the hypotensive drugs, and displays the medication treatment result information on the doctor terminals DT1 to DTm. Accordingly, the doctor can check the selection order of the antihypertensive drugs at a glance, thereby making a further accurate determination on the next medication.

Further, in accordance with the request information sent from the doctor terminals DT1 to DTm in response to a request from the doctor, the medication support server SV generates the medication guide information indicating the type and amount of the antihypertensive drug in consideration to the medication treatment result information corresponding to the target patient, and displays the medication treatment result information on the doctor terminals DT1 to DTm. Accordingly, when vacillating on the types and amounts of medicine to be prescribed, the doctor can refer to the medication guide information to make a determination on the details of the medication.

### Other Embodiments

In the first embodiment, the medication support server SV sets the granularity of the time axis in the medication treatment result information to “week” as an example. However, the granularity of the time axis may be set to “day” or “month”. Alternatively, the granularity of the time axis may be arbitrarily set by the doctor at the doctor terminals DT1 to DTm.

In addition, the medication support server SV arbitrarily changes and sets the layout of the blood pressure (BP) transition 41, the activity amount (Activity) change 42, the drug-taking adherence (Adherence) 43, and the medication management information 44 in the medication treatment result information, in response to a doctor's request or the like sent from the doctor terminals DT1 to DTm. Furthermore, when the medication support server SV generates the medication management information 44, the shape of the block representing one unit of the antihypertensive drug is not limited to a square, but may be a circle or capsule shape reminiscent of a general tablet shape. In addition, the types of antihypertensive drugs may be represented by differentiating block shapes instead of color and gray scale.

Furthermore, in the first embodiment, when generating the medication treatment result information, the medication support server SV displays the blood pressure (BP) transition 41, the activity amount (Activity) change 42, the drug-taking adherence (Adherence) 43, and the medication management information 44 in association with one another in the time line. Alternatively, the medication support server SV may display only the drug-taking adherence (Adherence) 43 and the medication management information 44 in association in the time line. In this way, the drug-taking adherence information is displayed in association with the information indicating the transition of the type and amount of the medicine in the time line. Accordingly, the drug-taking adherence can be considered as requirements of determination on medication for the patient, which allows a further efficient and accurate determination on medication in consideration to the drug-taking status of the patient.

Furthermore, in the first embodiment described above, the medication support server SV displays a display screen indicating medication candidates and the like on the doctor terminals DT1 to DTm as an example. Alternatively, the display screen may be displayed on a terminal used by another prescriber such as a pharmacist instead of the doctor terminals. In addition, the configuration and installation location of the medication support apparatus, the procedure and contents of the medication support processing, and the like can be variously modified and implemented without departing from the scope of the present invention.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

Some or all of the above-mentioned embodiments can be described as in the following additional statements but are not limited to the following additional statements.

### (Supplementary Statement 1)

A medication support apparatus (SV) capable of communicating via a network (NW) between a first terminal (DT1 to DTm) used by a prescriber of medicine and a second terminal (UT1 to UTn) connected to a blood pressure monitor (BT1 to BTn) or containing the blood pressure monitor and used by a patient, the medication support apparatus (SV) including at least one hardware processor (1) and a memory (2) connected to the hardware processor (1), in which

the hardware processor (1)

acquires medication information including types and amounts of medicine prescribed for the patient from the first terminal (DT1 to DTm) via the network (NW),

acquires blood pressure measurement information of the patient obtained by the blood pressure monitor (BT1 to BTn) from the second terminal (UT1 to UTn) via the network (NW),

generates medication treatment result information for the patient to display the types and amounts of medicine included in the acquired medication information and transition of blood pressure value based on the acquired blood pressure measurement information in a time line in association with each other, based on information indicating medication date and time and measurement date and time respectively included in the medication information and the blood pressure measurement information, and

transmits the generated medication treatment result information to the first terminal (DT1 to DTm) as the request source via the network (NW).

### (Supplementary Statement 2)

A medication support apparatus (SV) capable of communicating via a network (NW) between a first terminal (DT1 to DTm) used by a prescriber of medicine and a second terminal (UT1 to UTn) connected to a blood pressure monitor (BT1 to BTn) or containing the blood pressure monitor and used by a patient, the medication support apparatus (SV) including at least one hardware processor (1) and a memory (2) connected to the hardware processor (1), in which

the hardware processor (1)

acquires medication information including types and amounts of medicine prescribed for the patient from the first terminal (DT1 to DTm) via the network (NW),

acquires blood pressure measurement information of the patient obtained by the blood pressure monitor (BT1 to BTn) from the second terminal (UT1 to UTn) via the network (NW),

acquires drug-taking information indicating drug-taking status of the patient from the second terminal (UT1 to UTn) via the network (NW),

generates, for the patient, drug-taking adherence information indicating a status of drug compliance based on the acquired drug-taking information, generates medication treatment result information to display the generated drug-taking adherence information and the types and amounts of medicine included in the acquired medication information in a time line in association with each other, based on medication date and time and drug-taking date and time respectively included in the medication information and the drug-taking information, and

transmits the generated medication treatment result information to the first terminal (DT1 to DTm) as the request source via the network (NW).

### (Supplementary Statement 3)

A medication support method executed by an apparatus capable of communicating via a network (NW) between a first terminal (DT1 to DTm) used by a prescriber of medicine and a second terminal (UT1 to UTn) connected to a blood pressure monitor (BT1 to BTn) or containing the blood pressure monitor (BT1 to BTn) and used by a patient, the apparatus including at least one hardware processor (1) and a memory (2), the medication support method including:

a medication information acquisition step of, by using the at least one hardware processor (1) and the memory (2), acquiring medication information including types and amounts of medicine prescribed for the patient from the first terminal (DT1 to DTm) via the network (NW);

a blood pressure measurement information acquisition step of, by using the at least one hardware processor (1) and the memory (2), acquiring blood pressure measurement information of the patient obtained by the blood pressure monitor (BT1 to BTn) from the second terminal (UT1 to UTn) via the network (NW);

a generation step of, by using the at least one hardware processor (1) and the memory (2), generating medication treatment result information for the patient to display the types and amounts of medicine included in the acquired medication information and transition of blood pressure value based on the acquired blood pressure measurement information in a time line in association with each other, based on information indicating medication date and time and measurement date and time respectively included in the medication information and the blood pressure measurement information; and

a transmission step of, by using the at least one hardware processor (1) and the memory (2), transmitting the generated medication treatment result information to the first terminal (DT1 to DTm) as the request source via the network (SV).

What is claimed is:

1. A medication support apparatus capable of communicating via a network between a first terminal used by a prescriber of medicine and a second terminal connected to a blood pressure monitor or containing the blood pressure monitor and used by a patient, the apparatus comprising:
a first acquisition unit configured to acquire medication information including types and amounts of medicine prescribed for the patient from the first terminal via the network;
a second acquisition unit configured to acquire blood pressure measurement information of the patient obtained by the blood pressure monitor from the second terminal via the network;
a third acquisition unit configured to acquire drug-taking information indicating a drug-taking status of the patient from the second terminal via the network;
a generation unit configured to generate, for the patient, drug-taking adherence information indicating a status of drug compliance based on the acquired drug-taking information, and generate medication treatment result information to display the types and amounts of medicine included in the acquired medication information, transition of blood pressure value based on the acquired blood pressure measurement information, and the generated drug-taking adherence information in a time line in association with one another, based on information indicating medication date and time, measurement date and time, and drug-taking date and time respectively included in the medication information, the blood pressure measurement information, and the drug-taking information; and
a transmission unit configured to transmit the generated medication treatment result information to the first terminal via the network.

2. The apparatus according to claim 1, wherein the generation unit is configured to detect a change exceeding a preset amount of the blood pressure value from the transition of the blood pressure value based on the blood pressure measurement information, and generate the medication treatment result information to display information indicating details of the change to be added to at a corresponding position in a time line of a graph indicating the transition of the blood pressure value.

3. The apparatus according to claim 1, wherein the generation unit is configured to generate the medication treatment result information to display the types of the medicine included in the medication information as blocks different in color or gray scale and display the amounts of the medicine by the number of stacks of the blocks.

4. The apparatus according to claim 1, further comprising a fourth acquisition unit configured to acquire activity amount information indicating an amount of activity of the patient from the second terminal via the network, wherein
the generation unit is configured to generate information indicating transition of the activity amount based on the acquired activity amount information, and generate the medication treatment result information to display the generated information indicating the transition of the activity amount in a time line in association with the types and amounts of the medicine and the transition of the blood pressure value.

5. The apparatus according to claim 1, further comprising a transmission unit configured to, when receiving a medication support request for the patient from the first terminal, refer to at least the medication treatment result information corresponding to the patient to generate medication guide information indicating candidates for types and amounts of medicine to be prescribed to the patient, and transmit the generated medication guide information to the first terminal as the request source via the network.

6. A medication support apparatus capable of communicating via a network between a first terminal used by a prescriber of medicine and a second terminal connected to a blood pressure monitor or containing the blood pressure monitor and used by a patient, the medication support apparatus comprising:
a first acquisition unit configured to acquire medication information including types and amounts of medicine prescribed for the patient from the first terminal via the network;
a third acquisition unit configured to acquire drug-taking information indicating a drug-taking status of the patient from the second terminal via the network;
a generation unit configured to, for the patient, generate drug-taking adherence information indicating a status of drug compliance based on the acquired drug-taking information, and generate medication treatment result information to display the generated drug-taking adherence information and the types and amounts of medicine included in the acquired medication information in a time line in association with each other based on medication date and time and drug-taking date and time respectively included in the medication information and the drug-taking information; and
a transmission unit configured to transmit the generated medication treatment result information to the first terminal via the network.

7. A medication support method executed by an information processing apparatus capable of communicating via a network between a first terminal used by a prescriber of medicine and a second terminal connected to a blood pressure monitor or containing the blood pressure monitor and used by a patient, the method comprising:
acquiring medication information including types and amounts of medicine prescribed for the patient from the first terminal via the network;
acquiring blood pressure measurement information of the patient obtained by the blood pressure monitor from the second terminal via the network;
acquiring drug-taking information indicating a drug-taking status of the patient from the second terminal via the network;
generating, for the patient, drug-taking adherence information indicating a status of drug compliance based on the acquired drug-taking information, and generating medication treatment result information to display the types and amounts of medicine included in the acquired medication information, transition of blood pressure value based on the acquired blood pressure measurement information, and the generated drug-taking adherence information in a time line in association with one another, based on information indicating medication date and time, measurement date and time, and drug-taking date and time respectively included in the medication information, the blood pressure measurement information, and the drug-taking information; and
transmitting the generated medication treatment result information to the first terminal via the network.

8. A non-transitory computer readable medium storing a computer program which is executed by a computer to provide the steps of, the computer being included in an information processing apparatus capable of communicating via a network between a first terminal used by a prescriber of medicine and a second terminal connected to a blood pressure monitor or containing the blood pressure monitor and used by a patient:
acquiring medication information including types and amounts of medicine prescribed for the patient from the first terminal via the network;
acquiring blood pressure measurement information of the patient obtained by the blood pressure monitor from the second terminal via the network;
acquiring drug-taking information indicating a drug-taking status of the patient from the second terminal via the network;
generating, for the patient, drug-taking adherence information indicating a status of drug compliance based on the acquired drug-taking information;
generating medication treatment result information to display the types and amounts of medicine included in the acquired medication information, transition of blood pressure value based on the acquired blood pressure measurement information, and the generated drug-taking adherence information in a time line in association with one another, based on information indicating medication date and time, measurement date and time, and drug-taking date and time respectively included in the medication information, the blood pressure measurement information, and the drug-taking information; and
transmitting the generated medication treatment result information to the first terminal via the network.
